(19) United States
(12) Patent Application Publication
(10) Pub. No.: US 2026/0369373 A1
(43) Pub. Date: Nov. 19, 2026

(54) METHOD FOR GENERATING A REPORT FOR A MEDICAL PROCEDURE

(71) Applicant: VULGAROO, BORDEAUX (FR)

(72) Inventor: CHRISTOPHE LELONG, BORDEAUX (FR)

(21) Appl. No.: 19/153,675

(22) PCT Filed: Feb. 6, 2024
(86) PCT No.: PCT/EP2024/052942

§ 371 Date: Aug. 5, 2025

(30) Foreign Application Priority Data
Feb. 8, 2023 (FR) 23155506.1

(51) Int. Cl. G16H 15/00

(52) U.S. Cl. CPC G16H 15/00

(57) ABSTRACT

The invention relates to a method for generating a report for a medical procedure performed on a patient by a healthcare professional; the method being implemented by a computer system comprising a first computer terminal, a processing server and a second computer terminal, the method comprising the following steps: generating a first report, referred to as the expert report, for the medical procedure on the first computer terminal; the processing server automatically generating at least a second report, referred to as the simplified report, on the basis of the first report; the processing server transmitting the first and second reports at least to the first computer terminal; the processing server and/or the first terminal generating a digital identifier associated with the patient and with the medical procedure, allowing the patient to access the first and second reports via the second computer terminal.

### TECHNICAL FIELD

The invention relates to the technical field of healthcare, and more specifically to the production of medical reports.

### PRIOR ART

Typically, during a patient's care pathway, one or more medical examinations are prescribed, such as a consultation with a specialist, a medical examination involving imaging or a biological examination. The practitioner conducting the examination generally writes an evaluation or report of the medical examination, optionally including observations or diagnostic findings. This evaluation is then transmitted, either electronically via the patient's medical record, or directly via the patient, to the next practitioner in the care pathway, and/or to the doctor coordinating the care pathway.

While this method does enable the various medical professionals involved to have access to the results of previous examinations and to monitor the patient effectively throughout the care pathway, it has the disadvantage of excluding the patient from the therapeutic strategy of the care pathway.

A medical report generally contains complex technical medical terms that the patient may not be familiar with. Furthermore, the answers and findings provided by the practitioner in the report are difficult for the patient to interpret, for example because the practitioner uses clinical terms or acronyms, or refers to values without providing a frame of reference for explanation.

This can lead to a loss of confidence in the proposed therapeutic strategy, and reduced compliance and participation in the care pathway, which in turn can lead to a failure of the care pathway.

There is thus a need for a solution that enables the exchange of medical reports between the various practitioners in a care pathway and the patient, according to adapted levels of comprehension.

The invention therefore takes place in this context and seeks to solve all of the aforementioned drawbacks while meeting said need.

### OVERVIEW OF THE INVENTION

To this end, the invention relates to a method for generating reports for a medical procedure performed on a patient by a healthcare professional; the method being implemented by a computer system comprising a first computer terminal, a processing server and a second computer terminal, the method comprising the following steps:
a. Generating a first report, referred to as the expert report, for the medical procedure on the first computer terminal;
b. The processing server automatically generating at least a second report, referred to as the simplified report, on the basis of the first report;
c. The processing server transmitting the first and second reports at least to the first computer terminal;
d. The processing server and/or the first terminal generating a digital identifier associated with the patient and with said medical procedure, allowing the patient to access said first and second reports via the second computer terminal.

The invention thus proposes, for example, that during the medical procedure, a healthcare professional writes the expert medical report using a computer terminal connected to a processing server. The medical procedure may be investigative or diagnostic, and may include a medical history, inspection, palpation, auscultation, sampling, examination involving medical imaging, or analysis of a sample and/or medical image. The medical procedure may also be a therapeutic procedure, involving an oral interview to provide therapeutic advice, the administration of medication, or a surgical, radiological or physical intervention. All or part of the medical procedure may be performed by doctors from different medical specialties, including surgeons, psychologists, cardiologists, oncologists and radiologists; by dentists, nurses, medical secretaries, pharmacists, physiotherapists, medical biologists, laboratory technicians, nurses' aids and ambulance drivers.

The first report can thus be transmitted from the computer terminal to the processing server, for example via a wireless communication protocol, or be written directly on a processing server software application accessible from the computer terminal. The first report is thus available and accessible from other computer terminals authorized to connect to the processing server, for example to enable another healthcare professional, such as another practitioner, the attending physician or other participants in the care pathway, to view this report.

At the same time, the processing server can generate one or more additional reports from the first report, each report corresponding to a given simplified comprehension level. After this or these simplified reports have been generated, the processing server can then transmit all the reports to the first computer terminal together with a digital identifier, such as a QR code, containing a link which, when scanned using a smartphone forming the second computer terminal, enables the patient to download the first and second reports onto a software application installed on this smartphone. Note that the digital identifier can be generated by the first computer terminal itself. This digital identifier can thus be communicated, if necessary, by the healthcare professional to the patient, either by showing him a monitor of the first computer terminal on which this digital identifier is displayed, or by printing a medium, such as a prescription, including this digital identifier.

The first expert report and each of the simplified reports can then be transmitted to the second computer terminal to be read by the patient. In this way, the patient is provided with different reports for the same medical procedure, each associated with a different comprehension level, thus improving the chances that the patient will understand the content of the first report, and thus remain involved in the care pathway.

### DEFINITIONS

In the present invention, “computer terminal” means, but is not limited to, a desktop computer, a laptop, a server, a touch-sensitive tablet, a smartphone, a cell phone and/or computer network equipment.

Preferably, each computer terminal is equipped with a communication unit capable of exchanging data with a communication unit of the processing server, notably using one or more wireless communication protocols.

If desired, the first computer terminal can be connected to or form part of one or more hospital computer systems to receive data relating to the medical procedure via other elements of this system, said computer system being, for example, a radiology information system (RIS) and/or a picture archiving and communication system (PACS).

In the present invention, the processing server can be part of the hospital IT system to which the first computer terminal belongs. If required, the method according to the invention can be implemented by means of software programs or applications, part of which can be installed on the first computer terminal, in particular for the purposes of generating the first report. The first report, once generated, will be transmitted to the processing server.

Alternatively, the processing server could be a remote server of such a hospital IT system, the services of said processing server then being accessible by the first IT terminal by means of cloud computing methods. If required, all the programs or software applications needed to implement the method according to the invention can be installed on the processing server, and some or all of these programs or software applications can be accessed from the first computer terminal via wireless communication protocols, such as an Internet browser and HTTPS and IP protocols. The first report will thus be generated directly on the remote server.

In this case, it may be envisaged that the method according to the invention includes a prior step of identification and/or authentication of the person and/or application in charge of generating the first report, in particular through a strong authentication method implemented by the processing server or another server dedicated to this method.

### EMBODIMENTS

In one embodiment of the invention, the step of generating the first report includes:
a. a step for selecting a report template from a library of predetermined report templates on the first computer terminal;
b. a step for completing a medical questionnaire associated with the selected report template on the first computer terminal;
c. and for each question of the questionnaire, a step for providing at least one response, via the first computer terminal, the first report including at least all the responses provided when completing the questionnaire.

For example, the first report could include only the set of responses provided when completing the questionnaire, each response being associated with a given question in the questionnaire, or the set of question-response pairs provided when completing the questionnaire.

Preferably, said template library can be stored in a memory of the processing server or of the first computer terminal, and the selection step can be implemented through a software application accessible from the first computer terminal on which said library is displayed, the software application being provided with an interface for searching and/or selecting a template.

For example, the method may include a preliminary step of identifying one or more characteristics of the medical procedure, such as a pathology, a type of examination, a practitioner's specialty or a medical imaging modality, on a software application, via the first computer terminal. If required, the step of selecting a report template may involve displaying one or more templates from the library in said software application, which templates are pre-selected according to said identified characteristic(s).

The step of completing said medical questionnaire may involve displaying the questionnaire on a software application accessible from the first computer terminal, or alternatively reading said questionnaire by a software application accessible from the first computer terminal and equipped with voice synthesis means.

In one example embodiment, the medical questionnaire could be a dynamic sequential questionnaire. In other words, the questions of the questionnaire can be completed sequentially, one after the other or in successive groups, with the completion of a question or group of questions depending on the responses provided to all or some of the preceding questions or groups of questions.

The responses to the questions can be provided manually by a person, in particular via the keyboard of the first computer terminal. Alternatively, responses can be dictated to the first computer terminal via a microphone, the first computer terminal being equipped with a dictation and voice recognition software application.

It may be envisaged for responses to be provided by one or more people, or even by one or more machine learning algorithms.

In this way, the first report can include all the question-response pairs provided when completing the questionnaire, which can be reordered and/or grouped into sections in the first report. This set may optionally be accompanied by a set of medical findings.

In another embodiment of the invention, it may be envisaged for the first report to be written manually by a healthcare professional, identical to or different from the one who performed the medical procedure, and then scanned using the first computer terminal. Alternatively, it may be envisaged for the first report to be written directly on the first computer terminal by a healthcare professional, identical to or different from the one who performed the medical procedure, for example using a text editor or a medical report editor.

In one embodiment of the invention, the processing server includes a memory wherein are stored, for each report template in the library, at least one predefined set of a plurality of rules for transforming a response in said report into a response and/or at least one predefined grid for selecting responses from the set of responses in said report. If necessary, the step of automatically generating the second report includes:
a. a step wherein the processing server selects, from its memory, the selection grid and/or the set of transformation rules associated with the template of the first report;
b. a step for the processing server to apply the selected selection grid and/or set of transformation rules to the first report to generate the second report.

Each transformation rule can be a rule for transforming a response associated with a given question into another response associated with this given question, a rule for transforming a question-response pair into another question-response pair, or a rule for transforming a response associated with a given question into another question-response pair. Equivalently, the selection grid may be a grid for selecting responses from among all the responses in said report, or a grid for selecting question-response pairs from among all the question-response pairs in said report.

This set of rules and this selection grid can be used to generate a second report adapted to a lower comprehension level than the first report, by simplifying the vocabulary or syntax used and deleting responses or question-response pairs that do not provide useful or essential information for the patient. Each set and each selection grid, for example, can be defined in advance by expert patients. The processing server may apply only a selection grid, without applying any transformation rules, or the processing server may apply only transformation rules, without applying a selection grid, or the processing server may apply both transformation rules and a selection grid.

Advantageously, the step of generating the first report includes, for each question of the questionnaire, a step of providing at least one response, via the first computer terminal, to form a question-response pair, the first report including all the questions-responses provided during the completion of the questionnaire; and, for each report template of the library, are stored in the memory of the processing server: at least one predefined set of a plurality of rules for transforming one question-response pair of said report into another question-response pair and/or at least one predefined grid for selecting question-response pairs from the set of question-response pairs of said report.

Advantageously, each rule of a set of transformation rules associated with a report template is able to replace, in at least one question-response pair of said report, at least one given input of the question and/or response of said pair with another predetermined input.

An “input” is a term, a proposition made up of several terms, a value or a Boolean of the “YES” or “NO” type. In other words, it is possible to transform all or part of a question and/or response to simplify the vocabulary used, to transform values into relative terms, to transform a question-response pair into a Boolean proposition or vice versa, to invert grammatical syntax, etc. It is also possible to synthesize several question-response pairs into a single simpler question-response.

If required, a set of transformation rules associated with a report template may include at least one rule able to replace, in a finding of said report, at least one given input of said finding with another predetermined input.

Advantageously, a selection grid associated with a report template is able to retain or delete each question-response pair of said report according to the question and/or response of said pair. For example, a question-response pair can be maintained depending on whether the response provided to a given question is positive or negative, or whether the value provided is outside or within a predefined range.

In one embodiment of the invention, for each report template in the library, at least one predefined set of a plurality of transformation rules and/or at least one predefined selection grid associated with a first comprehension level and at least one predefined set of a plurality of transformation rules and/or at least one predefined selection grid associated with a second comprehension level are stored in the memory of the processing server. If necessary, the automatic generation step includes:
a. a step wherein the processing server selects, from its memory, the selection grid and/or the set of transformation rules associated with the template of the first report and the first comprehension level and the selection grid and/or the set of transformation rules associated with the template of the first report and the second comprehension level;
b. a step wherein the processing server applies, to the first report, the selection grid and/or the set of transformation rules associated with the first comprehension level to generate the second report and the selection grid and/or the set of transformation rules associated with the second comprehension level to generate a third report; and in that the transmission step includes the transmission, by the processing server, of the first, second and third reports to at least the first computer terminal.

This means that multiple reports of the same medical procedure can be generated at different levels of comprehension, enabling patients to progress in their understanding of the care pathway they are following.

For example, the second report may be a detailed lay report, comprising all the responses or question-response pairs from the first report, and wherein technical and/or medical terms are replaced by common terms; and the third report may be an abbreviated lay report, comprising only those responses or question-response pairs from the first report that have been deemed essential for the patient, and wherein technical and/or medical terms are replaced by common terms.

In a further, alternative or cumulative embodiment of the invention, the processing server includes a memory wherein at least one lexicon comprising a plurality of predetermined keywords and at least one dictionary associating each keyword in the lexicon with a definition of said keyword are stored. If necessary, the step of automatically generating the second report may include:
a. a step wherein the processing server identifies one or more keywords in the first report from said lexicon;
b. for each identified keyword, a step wherein the processing server selects a definition associated with said identified keyword from the dictionary;
c. a step wherein the processing sector inserts each selected definition into the first report to generate the second report.

The second report thus corresponds to the first report, enriched with definitions of medical, scientific or technical terms, to make it easier for the patient to understand. This method is particularly suited to cases where the first report is generated without using a questionnaire associated with a pre-established report template, and in particular when it is freely entered by the healthcare professional using a text editor, or when it is written manually, or when it is dictated.

Preferably, the generating step may comprise, prior to the keyword identification step, a step of extracting text from the first report and/or a step of recognizing text in the first report, in particular implemented using optical character recognition methods. If necessary, the keyword identification step will be implemented based on texts extracted from the first report and/or recognized in the first report.

Advantageously, the keyword identification step to comprise comparing each of the terms in the first report with the keywords in the lexicon and/or searching the terms in the first report for each of the keywords in the lexicon. If necessary, said comparison and/or said search may extend to the identification of terms related and/or similar to said keywords and/or synonyms of said keywords.

Advantageously, each selected definition can be inserted as a footnote in the second report or in an appendix to the second report, and each keyword identified in the first report can be cross-referenced to this definition in the second report. Alternatively, each keyword identified in the first report can be transformed, in the second report, into a hyperlink providing access to the selected definition of said keyword. Also alternatively, each keyword identified in the first report can be accompanied by a listener for a given event, such as a click on the keyword or a hover over the keyword. If necessary, the triggering of said given event by the patient, via the second computer terminal, may cause the selected definition of said keyword to be displayed, for example in a tooltip.

Advantageously, at least one first dictionary associated with a first comprehension level and at least one second dictionary associated with a second comprehension level are stored in the memory of the processing server. If necessary, the selection step includes, for each keyword identified, the selection of a definition associated with said keyword in the first dictionary and in the second dictionary; and the insertion step includes inserting each definition selected in the first dictionary into the first report to generate the second report and inserting each definition selected in the second dictionary into the first report to generate a third report; the transmission step comprises the transmission, by the processing server, of the first, second and third reports to at least the first computer terminal. Other dictionaries may also optionally be envisaged to obtain, for a keyword in a given language, a definition of said keyword in a different language.

This means that multiple reports of the same medical procedure can be generated at different levels of comprehension, enabling patients to progress in their understanding of the care pathway they are following.

In still another embodiment, the processing server includes a computing unit capable of implementing at least one machine learning algorithm trained to generate a simplified report from an expert report on a medical procedure. If necessary, the step of automatically generating the second report includes the processing server providing the computing unit with the first report as input to said machine learning algorithm, the simplified report generated in response by the machine learning algorithm forming the second report.

It may be envisaged that said machine learning algorithm comprises a large language model (LLM) and/or a recurrent neural network (RNN) comprising short-and long-term memory cells (LSTM) and/or gated recurrent units (GRU) and/or a transformer-type architecture, that is, having an encoder stage and a decoder stage each equipped with attention layers, or even a generative pre-trained transformer (GPT).

Said algorithm can be trained, or refined, using a training dataset comprising a plurality of pairs, in particular 1000 pairs, of expert and simplified medical reports. Each pair could, for example, be made up of an expert medical report generated by a doctor and a simplified report generated by the doctor, another healthcare professional or a patient association, based on the expert medical report. Alternatively or cumulatively, certain pairs of the training set can be generated automatically, using data augmentation methods, for example by replacing a word in a report with a synonym from a thesaurus and/or by shuffling the order of sentences in the report and/or by automatically translating the report into another language and then translating the translation back into the original language.

It may optionally be envisaged to pre-process each report for each pair in the training set, for example by substituting the corresponding word for each abbreviation and acronym, by replacing words identified as belonging to complex medical terminology with a definition from a medical dictionary, and/or by vectorizing the words in the report (also known as word embedding) using a pre-defined, pre-trained vectorization model.

Advantageously, the first report can be provided as a single input to said machine learning algorithm (zero-shot prompting method). Alternatively, the method may comprise a sub-step of constructing a prompt containing the first report and at least one example of a pair of an expert medical report and a simplified medical report, said prompt then being supplied as input to said machine learning algorithm (few-shot prompting method). Alternatively, or cumulatively, the prompt can integrate elements selected from a knowledge database according to a semantic similarity criterion with the first report (RAG method, or retrieval augmented generation).

Advantageously, the computing unit is able to implement a plurality of separate machine learning algorithms, each trained to generate a simplified report from an expert report of a medical procedure. If necessary, the step of automatically generating the second report includes the processing server providing the computing unit with the first report as input to each machine learning algorithm, and selecting a report from the set of simplified reports generated in response by the machine learning algorithms, the selected simplified report forming the second report. In particular, machine learning algorithms can be based on distinct architectures and/or have a common architecture but distinct hyperparameters. In particular, it may be envisaged that the step of automatically generating the second report includes calculating, for each simplified report generated, a value of a predetermined metric, the selection of said report being based on the values of the metric of said generated reports.

It may also be envisaged that information relating to the expected comprehension level of the simplified report is also provided as input to said machine learning algorithm, said information being inserted into the prompt, for example, and/or that the computing unit is able to implement several distinct machine learning algorithms, each trained to generate a simplified report according to distinct comprehension level from an expert report of a medical procedure.

In the above examples, comprehension levels could, for example, be defined according to Flesch-Kincaid readability tests or any other comprehension assessment method adapted to the field of medical reports.

In one embodiment of the invention, the method comprises, subsequent to the generation of the digital identifier:
a. a step wherein the patient inputs said digital identifier via the second computer terminal;
b. following said input; a step wherein the processing server transmits the first and second reports to the second computer terminal.

Advantageously, the second computer terminal can be equipped with a software application comprising an interface for selecting a comprehension level of the user of the second computer terminal. If necessary, the method includes a subsequent step of displaying, on said software application, the first report and/or the second report according to the comprehension level selected by the user of the second computer terminal by means of said selection interface. The patient can then read any of the reports to check and improve his comprehension level.

Advantageously still, said software application can include an interface for confirming comprehension by the user of the second computer terminal. Where appropriate, the method comprises:
a. Following the display, on said software application, of the first report and/or the second report, a step of displaying, on said software application, said comprehension confirmation interface;
b. Following the input of information by the user of the second computer terminal, via said comprehension confirmation interface, the transmission of said input information to the processing server and/or to the first computer terminal.

In this way, patients can confirm that they have read and understood one of the reports, or that none of the reports have enabled them to understand the results of the medical procedure, or that the reports lack clarity and that part of the report is unclear. This information is then transmitted to the processing server, or to the first computer terminal, so that the practitioner in charge of the medical procedure, or another participant in the care pathway, can take note of it and prepare a subsequent meeting with the patient to provide explanations.

The invention also relates to a computer program comprising program code which is designed to implement the method according to the invention.

The invention also relates to a data carrier on which the computer program according to the invention is recorded.

### BRIEF DESCRIPTION OF THE FIGURES

Other advantages and features of the present invention are now described with the aid of examples that are purely illustrative and in no way limit the scope of the invention, and based on the attached figures, wherein:

FIG. 1 shows, schematically and partially, a computer system for implementing a method according to an example embodiment of the invention;

FIG. 2 shows, schematically and partially, a method for generating reports of a medical procedure according to an example embodiment of the invention, implemented by means of the computer system shown in FIG. 1;

FIG. 3 shows, schematically and partially, a view of a medical questionnaire displayed on a computer terminal of the system of FIG. 1 during the implementation of a step of the method of FIG. 2;

FIG. 4 shows, schematically and partially, a medical report transmitted to a processing server of the system of FIG. 1 during the implementation of another step of the method of FIG. 2;

FIG. 5 shows, schematically and partially, a set of rules used by the processing server of the system of FIG. 1 when implementing another step of the method of FIG. 2;

FIG. 6 shows, schematically and partially, a selection grid used by the processing server of the system of FIG. 1 when implementing another step of the method of FIG. 2;

FIG. 7 shows, schematically and partially, different medical reports generated by the processing server of the system of FIG. 1 during the implementation of another step of the method of FIG. 2; and

FIG. 8 shows, schematically and partially, a view of an interface of a software application displayed on a computer terminal of the system of FIG. 1 during the implementation of another step of the method of FIG. 2.

In the following description, identical elements, by structure or function, appearing in different figures retain, unless otherwise specified, the same references.

The methods described here can also be implemented by software programs that can be run on a computer system. Additionally, they can be implemented by distributed and/or parallel processing, in particular for parallel processing of several data items.

The figures described herein are intended to provide a general understanding of the invention in various embodiments. These figures are not intended to serve as a complete description of all the elements and features of the devices, processors and systems required for the invention. Many other embodiments of the invention, or combinations thereof, may be apparent to the skilled person from reading this description, by combining the disclosed embodiments. Other embodiments can be derived from the description, so that structural and logical substitutions and changes can be made without departing from the scope of the present invention.

Additionally, the description and figures are to be regarded as illustrative rather than restrictive, and the appended claims are intended to cover all modifications, improvements and other embodiments of the invention. Thus, the scope of the following claims must be determined by the broadest possible interpretation of the claims and their equivalents, and must not be restricted or limited by the preceding description.

### DESCRIPTION OF THE EMBODIMENTS

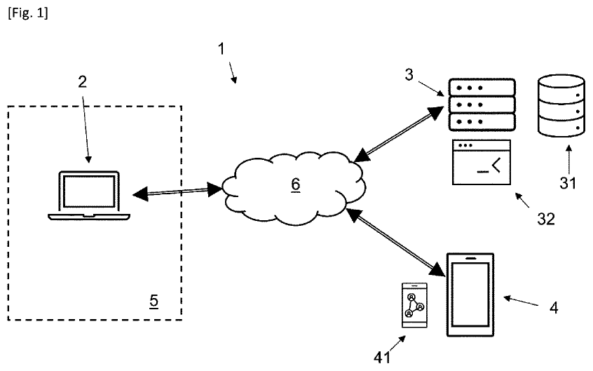

FIG. 1 shows a computer system 1 for implementing a method of generating reports for a medical procedure according to an example embodiment of the invention.

The computer system 1 includes a first computer terminal 2, a processing server 3 and a second computer terminal 4.

In the example described, the first computer terminal 2 is a desktop computer, part of a hospital information system 5, for example in a hospital, clinic, radiology unit, surgery unit, consulting room or analysis laboratory.

The processing server 3 is remote from the hospital information system. It includes a memory 31. The first computer terminal 2, or hospital information system 5, and the processing server 3 are each equipped with a wireless communication unit, so that the services offered by the processing server 3 can be accessed by the first computer terminal 2, notably via a wireless communication network 6 implementing HTTPS and IP-type communication protocols.

As will be described later, a software application 32 installed on the processing server 3 can in particular be run on the first computer terminal 2 via an Internet browser.

In the example described, the second computer terminal 4 is a patient's smartphone, which can receive, via a software application 41 installed on this smartphone, data transmitted by the processing server 3.

Alternatively, the first computer terminal 2, the hospital information system 5 and the processing server 3 can be housed in the same infrastructure, in which case they can be linked together by a wired and/or wireless communication network.

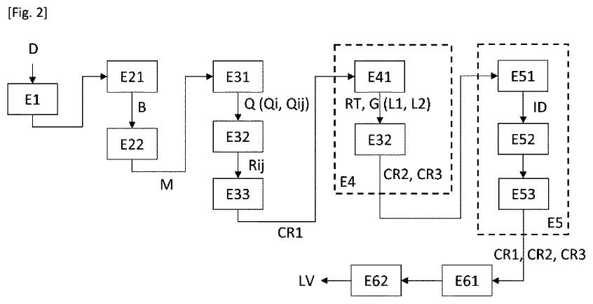

In connection with FIG. 2, a method will now be described for generating reports for a medical procedure performed by a healthcare professional on a patient, and implemented by means of the computer system 1.

In steps which are not shown, the patient follows a pathway within the hospital or laboratory where the medical procedure is performed. This pathway may include a reception step, one or more examination steps involving medical imaging, sampling, analysis, surgery, care or meetings with medical staff, and a discharge step. A set of data D can thus be collected during the course of the pathway by different components of information system 5, which may be medical staff, such as reception staff, an ambulance driver, a doctor or a nurse, medical equipment or a related information system such as a radiology information system (RIS), a picture archiving and communication system (PACS), or even through external information systems, such as the IT departments of public entities.

This data set D may thus comprise information from a computerized patient record, clinical data, medical images acquired according to one or more medical imaging modalities.

During one of the steps of the patient's pathway, a component of the information system 5 initiates the implementation of the method according to the invention. In the example described, this is the practitioner who performed the medical procedure on the patient. Alternatively, the method can be initiated by a non-human component of the information system 5, in particular by a computer program, or the method can be implemented by several components of the information system 5, in particular by different members of the medical staff and at different times in the patient's pathway, both upstream and downstream of the medical procedure.

In a first step E1, the practitioner connects via the first computer terminal 2 to the software application 32 of the remote server 3, through a strong authentication method implemented by another server dedicated to this method (not shown).

In a second step E21, a library B of report templates, stored in the memory 31 of the processing server 3, is displayed in the software application 32 on the first computer terminal 2, with an interface for searching and selecting a template. In particular, the library B can be filtered during display using all or some of the data in the data set B. Alternatively, the library B can be stored in a memory of another server of the computer system 1, and the processing server 3 can then query or access this other server to extract all or part of this library B.

In step E22, the practitioner thus selects a report template M on the first computer terminal 2, for example by searching the library B for a report template associated with the type of medical procedure he has performed, the medical imaging modality he has used and/or the pathology he has diagnosed.

In a step E31, a medical questionnaire Q, stored in the memory 31 and associated with the selected template M, is run on the software application 32. Alternatively, the questionnaire Q can be stored in the memory of another server in the computer system 1, and the processing server 3 can then query or access this other server to retrieve the questionnaire Q.

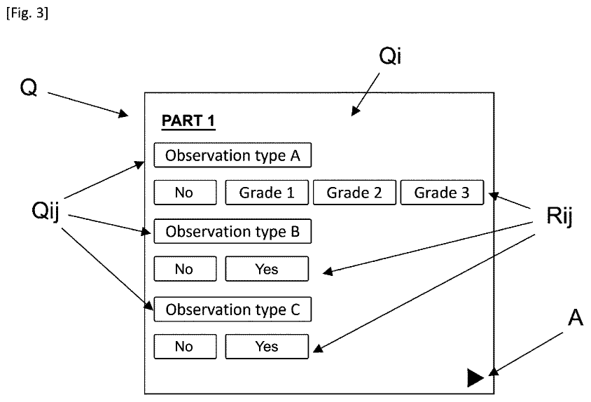

As shown in FIG. 3, in the example described, the questions Qij from the questionnaire Q are displayed in blocks or sections Qi on the first computer terminal 2 via the software application 32, with an action component A enabling the practitioner to navigate from one block to another on the application 32.

Each question Qij is displayed in the software application 32 with a response component enabling the practitioner to provide a response Rij to that question Qij in a step E32. In a non-limiting way, each response component Rij can indifferently comprise one, several or a combination of several of the following elements: a text input field, a selection button, a checkbox, a drop-down selection list, a cursor.

It may be envisaged that the medical questionnaire Q is dynamic, and that the display of a question Rij or even a block Qi depends on the answers provided to all or some of the preceding questions or blocks of questions, without departing from the scope of the present invention.

It may also be envisaged that all or some of the responses Rij are entered by other human, software or hardware components of the information system 5, during the patient's pathway, upstream and/or during and/or downstream of the medical procedure, in particular from the data set D.

In variants of the invention that are not shown, the questionnaire Q can be completed, in whole or in part, in other ways, in particular by being read by voice synthesis means on the first computer terminal 2 and/or the processing server 3. The responses Rij can be entered manually by a person, via a keyboard and/or mouse on the first computer terminal 2, or dictated, via a microphone on the first computer terminal 2.

In a non-limiting way, at the end of step E32, the practitioner can be asked to provide a set of diagnostic findings.

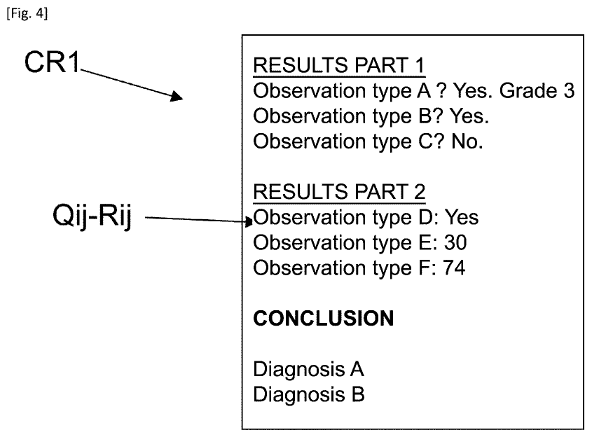

In a step E33, all the question-response pairs Qij-Rij provided at the end of steps E31 and E32, and any findings provided, are assembled by the processing server 3 to form a first report CR1, visible in FIG. 4,

In step E4, further reports CR2, known as lay reports, and CR3, known as simplified reports, are generated by the processing server 3 from the first report CR1. The various reports CR1, CR2 and CR3 can be generated simultaneously from the question-response pairs Qij-Rij without departing from the scope of the present invention.

To this end, for each report template M from the library B, at least one predefined set of a plurality of transformation rules RT and/or at least one predefined selection grid G associated with a first comprehension level L1 and at least one predefined set of a plurality of transformation rules RT and/or at least one predefined selection grid G associated with a second comprehension level L2 are stored in the memory 31 of the processing server s.

In the example described, the same set of transformation rules RT is associated with the two levels of comprehension L1 and L2, and no grid is associated with the first level L1, while a grid G is associated with the second level L2. Alternatively, different sets and grids can be associated with each of the levels L1 and L2, or a greater number of comprehension levels, without departing from the scope of the present invention. Comprehension levels can be predetermined, or chosen by the practitioner.

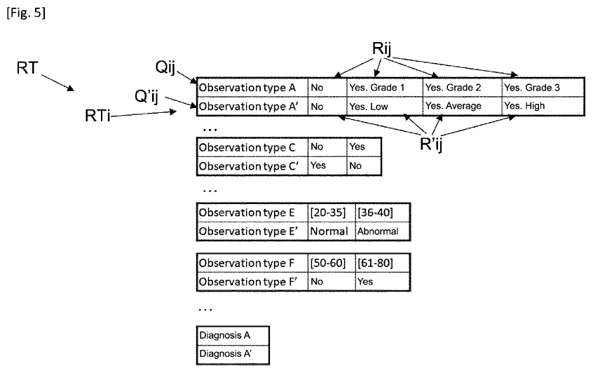

An example of a set of transformation rules RT is shown schematically in FIG. 5 in the form of a lookup table comprising a plurality of rules RTi.

Each rule RTi of the set of transformation rules RT associated with the report template M can thus replace, in each of the question-response pairs Qij-Rij of the first report CR1 and, where applicable, in the findings, at least one given input of the question Qij with another predetermined input to obtain another question Q′ij and at least one given input of the response Rij provided with another predetermined input to obtain another response R′ij.

As shown in FIG. 5, one or more terms, a value or a Boolean of the “YES” or “NO” type of a question Qij or a response Rij can be replaced indifferently with one or more terms, a value or a Boolean of the “YES” or “NO” type, so as to obtain a question Q′ij or a response R′ij whose syntax or vocabulary is simplified, whose values are transformed into relative terms, to transform a question-response pair into a Boolean proposition or vice versa, or even to invert a grammatical syntax.

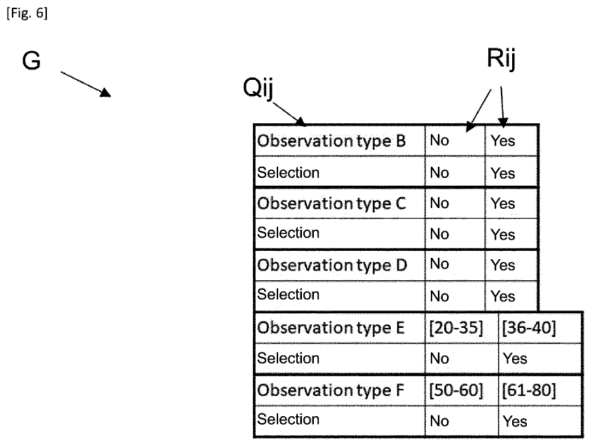

An example of a selection grid G in the form of a lookup table is shown schematically in FIG. 6.

This selection grid G, combined with the report template M, makes it possible to retain or delete certain question-response pairs Qij-Rij from the first report CR1, based on the responses Rij.

FIG. 5 shows that some of the question-response pairs Qij-Rij are retained depending on whether the response Rij provided to question Qij is positive or negative, or whether the value provided is in one range or another. It may be envisaged that a response Rij provided to a question Qij leads to the retention or deletion of another question-response pair Qij-Rij, or that responses Rij provided to several questions Qij lead to the retention or deletion of one or more of these questions-responses Qij-Rij.

Thus, in a selection step E41, the processing server 3 selects from its memory 31 the selection grids G and/or transformation rule sets RT associated with the selection report template M and the different comprehension levels L1 and L2.

Then, in an application step E42, for each of the comprehension levels L1 and L2, the processing server 3 applies the selection grid G and/or the transformation rule set RT associated with this comprehension level to generate another report.

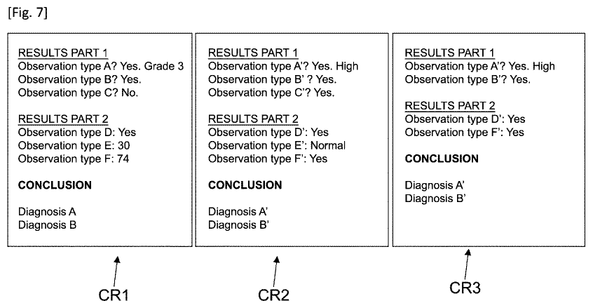

In the example described, as shown in FIG. 7, at the end of step E42, the processing server 3 generates a second lay report CR2, comprising question-response pairs Q′ij-R′ij obtained from all the question-response pairs Qij-Rij of the first report CR1 and the transformation rule set R. This second report CR2 thus corresponds to the first report CR1, wherein technical and/or medical terms are replaced by common terms.

At the end of step E42, the processing server 3 also generates a third, simplified report CR3, comprising question-response pairs Q′ij-R′ij obtained from only some of the question-response pairs Qij-Rij of the first report CR1, the selection grid G and the transformation rule set R. This third report CR3 comprises only those question-response pairs from the first report CR1 that have been deemed essential for the patient, and wherein technical and/or medical terms are replaced by common terms.

Note that the first report CR1 is automatically associated with a comprehension level of LO.

In step E5, the first, second and third reports CR1, CR2 and CR3 are transmitted by the transmission server 3 to the first and second computer terminals 2 and 4.

More precisely, in a step E51, the processing server 3 generates a digital ID, for example in the form of a QR code, which it transmits to the first computer terminal 2, so that it can be communicated to the patient. This QR code can either be scanned directly by the patient using the smartphone 4, or printed by the practitioner, for example on a prescription sheet, so that it can be scanned later by the patient using the smartphone 4. At the same time, the first, second and third reports CR1, CR2 and CR3 are transmitted by the transmission server 3 to the first computer terminal 2, via the software application 32.

In a step E52, the QR code ID is entered by the patient on the second computer terminal 4, for example by being scanned via the software application 41 installed on the smartphone 4.

In step E53, this QR code enables the patient to automatically download the reports CR1, CR2 and CR3 from the processing server 3 into the software application 41 installed on the smartphone 4.

In an alternative which is not shown, the first, second and third reports CR1, CR2 and CR3 can be transmitted by the transmission server 3 to another server equipped with a memory for storing computerized patient files. The patient can then access these reports by connecting via the second computer terminal 4 to a software application hosted by this other server.

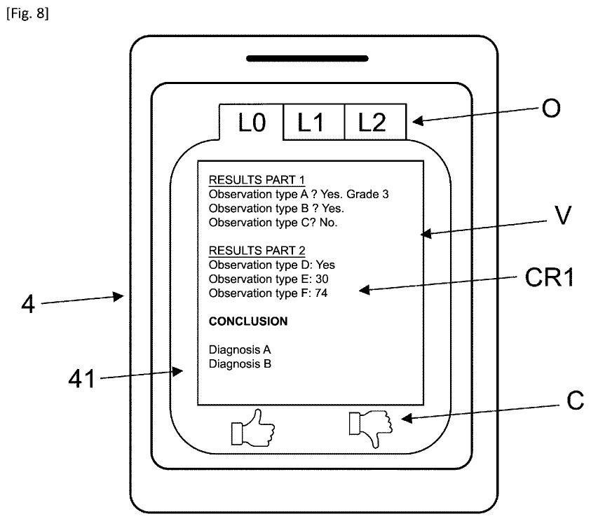

As shown in FIG. 8, the software application 41 includes an interface V for viewing said reports CR1, CR2 and CR3, as well as an interface O for selecting comprehension levels LO, L1 and L2.

In this way, the patient can select the comprehension level he feels is appropriate, via the selection interface O, and the corresponding report is displayed in step E61 in the viewing interface V.

The software application also includes a confirmation interface C, displayed simultaneously in step E61, enabling the patient to specify his comprehension level LV of the report displayed in this viewing interface V, after reading it. It may be envisaged that the confirmation interface C will allow the patient to select his comprehension level more precisely than that shown in FIG. 8 and/or add comments, without going beyond the scope of the present invention.

Thus, following an interaction between the patient and the confirmation interface C, the selected comprehension level is transmitted, in a step E62, to the processing server 3, and possibly relayed to the first computer terminal 2, so that the healthcare professional having performed the medical procedure can schedule an explanatory meeting with the patient if necessary. Alternatively, or cumulatively, the various reports CR1, CR2 and CR3, together with the selected comprehension level, can be transmitted to another computer terminal on the system 1, in particular one used by another person involved in the patient's care pathway.

It should be noted that different aspects of the various interfaces shown may be envisaged and/or other functionalities added, without departing from the scope of the present invention.

The preceding description clearly explains how the invention makes it possible to achieve the objectives it has set itself, namely to propose a method allowing the exchange of medical reports between the various practitioners in a care pathway and the patient, according to adapted levels of comprehension. These objectives are achieved in particular by means of a processing server capable of generating simplified, abridged and/or lay reports from a medical report generated at a first computer terminal, and transmitting all the reports to another computer terminal.

In any case, the invention is not limited to the embodiments specifically described in this document, and extends in particular to any equivalent means and to any technically operative combination of these means.

1. A method for generating reports for a medical procedure performed on a patient by a healthcare professional; the method being implemented by a computer system comprising a first computer terminal processing server and a second computer terminal, the method comprising the following steps:
a. Generating a first report, referred to as the expert report, for the medical procedure on the first computer terminal;
b. The processing server automatically generating at least a second report, referred to as the simplified report, on the basis of the first report;
c. The processing server transmitting the first and second reports at least to the first computer terminal;
d. The processing server and/or the first terminal generating a digital identifier associated with the patient and with said medical procedure, allowing the patient to access said first and second reports via the second computer terminal.

2. The method according to claim 1, wherein the step of generating the first report includes a step for selecting a report template from a library of predetermined report templates on the first computer terminal, a step for completing a medical questionnaire associated with the selected report template on the first computer terminal, and for each question of the questionnaire, a step for providing at least one response, via the first computer terminal, the first report including at least all the responses provided when completing the questionnaire.

3. The method according to claim 2, characterized in that the processing server includes a memory wherein are stored, for each report template in the library, at least one predefined set of a plurality of rules for transforming a response in said report into another response and/or at least one predefined grid for selecting responses from the set of responses in said report, and in that the step of automatically generating the second report includes:
a. a step wherein the processing server selects, from its memory, the selection grid and/or the set of transformation rules associated with the template of the first report;
b. a step for the processing server to apply the selected selection grid and/or set of transformation rules to the first report to generate the second report.

4. The method according to claim 3, wherein the step of generating the first report includes, for each question of the questionnaire, a step of providing at least one response, via the first computer terminal, to form a question-response pair, the first report including all the questions-responses provided during the completion of the questionnaire; and in that, for each report template of the library, are stored in the memory of the processing server: at least one predefined set of a plurality of rules for transforming one question-response pair of said report into another question-response pair and/or at least one predefined grid for selecting question-response pairs from the set of question-response pairs of said report.

5. The method according to claim 4, characterized in that each rule of a set of transformation rules associated with a report template is able to replace, in at least one question-response pair of said report, at least one given input of the question and/or response of said pair with another predetermined input.

6. The method according to claim 4, characterized in that a selection grid associated with a report template is able to retain or delete each question-response pair of said report according to the question and/or response of said pair.

7. The method according to claim 3, characterized in that, for each report template in the library, at least one predefined set of a plurality of transformation rules and/or at least one predefined selection grid associated with a first comprehension level and at least one predefined set of a plurality of transformation rules and/or at least one predefined selection grid associated with a second comprehension level are stored in the memory of the processing server; in that the automatic generating step includes:
a. a step wherein the processing server selects, from its memory, the selection grid and/or the set of transformation rules associated with the template of the first report and the first comprehension level and the selection grid and/or the set of transformation rules associated with the template of the first report and the second comprehension level;
b. a step wherein the processing server applies, to the first report, the selection grid and/or the set of transformation rules associated with the first comprehension level to generate the second report and the selection grid and/or the set of transformation rules associated with the second comprehension level to generate a third report; and in that the transmission step includes the transmission, by the processing server, of the first, second and third reports to at least the first computer terminal.

8. The method according to claim 1, characterized in that the processing server includes a memory wherein at least one lexicon comprising a plurality of predetermined keywords and at least one dictionary associating each keyword in the lexicon with a definition of said keyword are stored and in that the step of automatically generating the second report includes:
a. a step wherein the processing server identifies one or more keywords in the first report from said lexicon;
b. for each identified keyword, a step wherein the processing server selects a definition associated with said identified keyword from the dictionary;
c. a step wherein the processing sector inserts each selected definition into the first report to generate the second report.

9. The method according to claim 8, wherein at least one first dictionary associated with a first comprehension level and at least one second dictionary associated with a second comprehension level are stored in the memory of the processing server, characterized in that the selection step includes, for each keyword identified, the selection of a definition associated with said keyword in the first dictionary and in the second dictionary; and in that the insertion step includes inserting each definition selected in the first dictionary into the first report to generate the second report and inserting each definition selected in the second dictionary into the first report to generate a third report; and in that the transmission step comprises the transmission, by the processing server, of the first, second and third reports to at least the first computer terminal.

10. The method according to claim characterized in that the processing server includes a computing unit capable of implementing at least one machine learning algorithm trained to generate a simplified report from an expert report on a medical procedure and in that the step of automatically generating the second report includes the processing server providing the computing unit with the first report as input to said machine learning algorithm, the simplified report generated in response by the machine learning algorithm forming the second report.

11. The method according to one of the claim 1, characterized in that it comprises, subsequent to the generation of the digital identifier:
a. a step wherein the patient inputs said digital identifier via the second computer terminal;
b. following said input; a step wherein the processing server transmits the first and second reports to the second computer terminal.

12. The method according to claim 1, wherein the second computer terminal is equipped with a software application comprising an interface for selecting a comprehension level of the user of the second computer terminal, characterized in that it includes a subsequent step of displaying, on said software application, the first report and/or the second report according to the comprehension level selected by the user of the second computer terminal by means of said selection interface.

13. The method according to claim 12, wherein said software application includes an interface for confirming comprehension by the user of the second computer terminal, characterized in that it includes:
a. Following the display, on said software application, of the first report and/or the second report, a step of displaying, on said software application, said comprehension confirmation interface;
b. Following the input of information by the user of the second computer terminal, via said comprehension confirmation interface, the transmission of said input information to the processing server and/or to the first computer terminal.
